(19) United States
(12) Patent Application Publication
(10) Pub. No.: US 2024/0228377 A9
(43) Pub. Date: Jul. 11, 2024

(54) GREEN HIGH STRENGTH CEMENT

(71) Applicants: Saudi Arabian Oil Company, Dhahran (SA); William Marsh Rice University, Houston, TX (US)

(72) Inventors: Peter Boul, Houston, TX (US); Devashish Salpekar, Houston, TX (US); Pulickel M. Ajayan, Houston, TX (US)

(21) Appl. No.: 17/972,053

(22) Filed: Oct. 24, 2022

(51) Int. Cl. C04B 20/10; C04B 7/36; C04B 7/44; C04B 14/02; C04B 28/06; C04B 28/14; C04B 28/18; C07C 1/04

(52) U.S. Cl. CPC C04B 20/1055; C04B 7/367; C04B 7/44; C04B 14/026; C04B 28/06; C04B 28/141; C04B 28/188; C07C 1/04

(57) ABSTRACT

A system and a method for generating carbon nanotube (CNT)-reinforced cementitious materials are provided. An exemplary method includes capturing carbon dioxide formed in while calcining cementitious precursors, converting the carbon dioxide to hydrocarbons, producing CNTs on the calcined cementitious precursors from the hydrocarbons, and forming CNT-reinforced, cementitious materials from the calcined cementitious precursors comprising the CNTs.

### TECHNICAL FIELD

The present disclosure is directed to techniques for capturing carbon dioxide during the production of cement and using it to form structures to strengthen the cement.

### BACKGROUND

Portland cement production involves heat treatment of calcite and silica at high temperatures (>1300° C.) to produce calcium silicate clinkers containing tricalcium and dicalcium silicates as shown in Equations 1 and 2.

CaCO³→CaO+CO₂ Eqn. 1

xCaO+SiO₂→CaₓSiOₓ₊₂ Eqn. 2

In Equations 1 and 2, x is 2 or 3. The reaction mechanism shown in Equation 1 releases a large amount of carbon dioxide (CO₂), emissions and contributes around 54% of the CO₂ emission from cement production. Moreover, due to the high reaction temperature, the usage of fossil fuels accounts for another 35% of the CO₂ emissions from cement production. On average, 1000 kg of cement production emits 927 kg of greenhouse gases (GHGs). With the global production of cement crossing 4 billion tons per year, the cement industry contributes around 7% of the total emission of global GHG with about 90% of these emissions coming from the production of clinker. Therefore, reducing GHG emissions from the production of cement will assist in reducing the overall production of GHGs. One of the common methods to reduce overall carbon footprint is by carbon capture and conversion.

### SUMMARY

An embodiment described herein provides a method for generating carbon nanotube (CNT)-reinforced cementitious materials. The method includes capturing carbon dioxide formed in while calcining cementitious precursors, converting the carbon dioxide to hydrocarbons, producing CNTs on the calcined cementitious precursors from the hydrocarbons, and forming CNT-reinforced, cementitious materials from the calcined cementitious precursors comprising the CNTs.

Another embodiment described herein provides a system for generating reinforced cementitious compositions. The system includes a tube reactor to calcine cementitious precursors, a tube furnace to form CNT-reinforced, cementitious materials from the calcined cementitious precursors and a hydrocarbon, a trap to capture carbon dioxide formed in the tube reactor, and a reactor to form the hydrocarbon from the carbon dioxide.

### BRIEF DESCRIPTION OF DRAWINGS

FIG. 1 is a schematic diagram of the formation of carbon dioxide during the pre-calcination of cementitious precursors in a tube furnace.

FIG. 2 is a schematic diagram of a reactor used to convert the CO₂ to low order hydrocarbons.

FIG. 3 is a schematic diagram of the production of CNTs on catalyst-impregnated, calcined cementitious precursors to form CNT-reinforced cementitious materials.

FIG. 4 is an electron micrograph of the CNTs formed on the cementitious material.

FIG. 5 is a schematic diagram of a combined system for making CNT reinforced CNT-reinforced, cementitious materials by capturing carbon dioxide formed during the process.

FIG. 6 is a schematic diagram of a system for producing CNT-reinforced, cementitious materials.

FIG. 7 is a process flow diagram of a method for generating CNT-reinforced cementitious materials from carbon dioxide captured during the production of the cementitious materials.

### DETAILED DESCRIPTION

There are two general techniques for carbon capture, direct air capture of carbon compounds, and the capture of carbon compounds as they are produced, for example, from the steel industry, power industry, cement production, and the like. While developments in direct air capture have improved the efficiency, capturing carbon compounds as they are produced is more practical, as the carbon compounds are at higher concentrations prior to being released into the atmosphere and a high demand for infrastructure resulting in increases in production indicates that capturing at the production source may provide a more rapid reduction in GHGs.

Nanomaterials are being studied for improving the properties of cementitious materials during the production or the hydration of the cementitious materials. Different carbon allotropes, including CNTs, are being tested for this purpose. CNTs are tubular structures of carbon in which the carbon is arranged in a hexagonal lattice around the circumference of the tubular structure. Different types of CNTs exist, including single walled CNTs and multi-walled CNTs. Either of these CNTs, or a mixture of both, can be formed during the production of cementitious precursors and used as the reinforcing material in cement.

CNTs have unique properties due to strong covalent bonding (sp2) between the carbon atoms in the structure. The hybridized structure leaves each carbon atom with a spare electron, resulting in high electrical and thermal conductivity relative to other forms of carbon. Moreover, strong covalent bonding between the carbon atoms provides the CNTs with high tensile strength (>100 GPa) and a high strength to weight ratio (>50000 kN m kg-1). Accordingly, the presence of CNTs in cement can significantly affect the properties, such as compressive strength, conductivity, and consecutively piezoelectricity, among others.

A simple way to mass-produce CNTs is using catalytic chemical vapor deposition, for example, growing the CNTs on the surface of the catalyst. For example, a carbon source in the presence of heat breaks into carbon atoms and hydrogen molecules, as shown in Equation 3.

CₓH_{y}→xC+y/2H₂ Eqn. 3

The carbon atoms then react with catalyst particles to grow the tubular structures. As can be seen in Equation 3, the growth of the carbon nanotubes provides a source of hydrogen, which may be used in other portions of the process, as described below.

Techniques provided in examples herein capture carbon compounds generated during the production of cementitious material from cementitious precursors. As used herein, cementitious precursors include tricalcium silicate (C3S), dicalcium silicate (C2S), calcium aluminoferrite (C4AF), or calcium sulfate dihydrate (C1, gypsum), or any combinations thereof, termed calcite/silica. Cementitious materials include materials that form cement during hydrolysis, such as alite and belite. The carbon compounds are used to form reinforcing materials on the cementitious materials. The reinforcing materials include nanomaterials, such as carbon nanotubes (CNTs). The CNT-reinforced, cementitious materials can then be used to make a reinforced cement. The techniques include three general actions, although these may be performed in single reactors as needed. Initially, the carbon dioxide produced during the production of calcined cementitious precursors is captured, for example, by adsorption or reaction. The captured carbon dioxide is then used to produce light hydrocarbons, such as methane. The light hydrocarbons are used to produce carbon nanotubes on catalyst particles deposited on the calcined cementitious precursors forming CNTs as the cementitious precursors are calcined to form the cementitious materials.

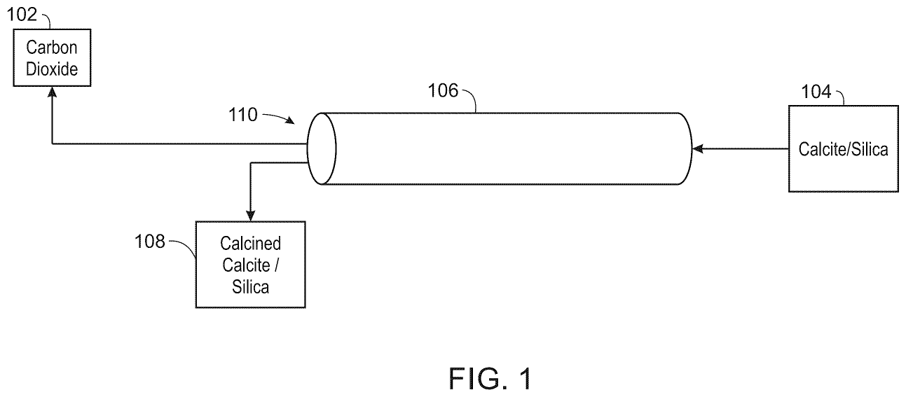

FIG. 1 is a schematic diagram of the formation of carbon dioxide 102 during the pre-calcination of cementitious precursors 104 in a tube reactor 106. The cementitious precursors 104 are pre-calcined at a temperature of about 800° C. to about 900° C., forming calcined cementitious precursors 108, e.g., calcined calcite/silica, and releasing the carbon dioxide 102. In some embodiments, the tube reactor 106 is tilted from the material entry to the material exit, and material is moved along the tube reactor 106 by rotating the tube reactor 106.

The carbon dioxide 102 can be captured in a CO₂ trap that comprises carbonaceous materials, metal oxides, dichalcogenides, nitrides, or halides, or any combinations thereof. The CO₂ trap is connected at the gas exhaust of the downstream end 110 of the tube reactor 106. In some embodiments, two CO₂ traps are coupled in parallel to the gas exhaust. One of the CO₂ traps can be capturing CO₂ from the process, while the other CO₂ trap is being heated to release the CO₂ to the next process unit. Alternating between the two CO₂ traps provides continuous operation of the system.

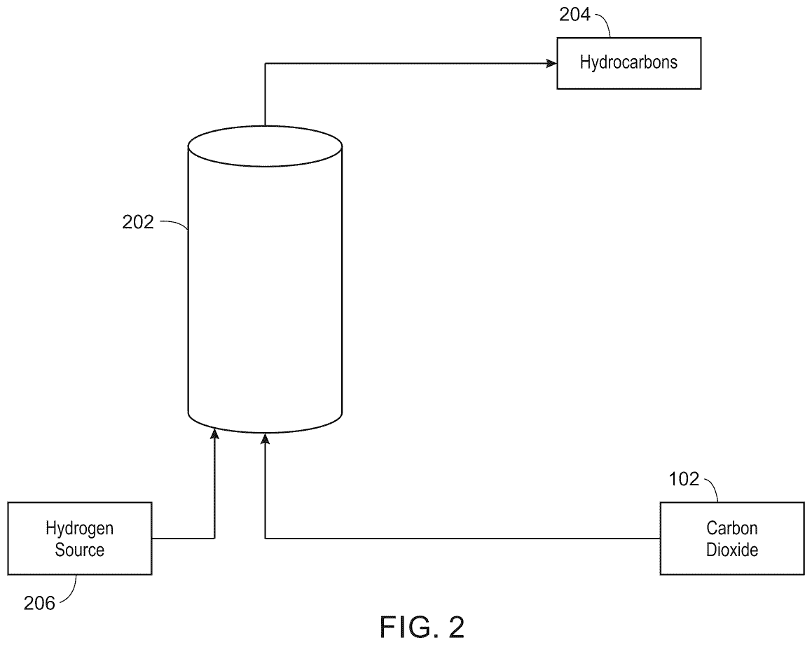

FIG. 2 is a schematic diagram of a reactor 202 used to convert the CO₂ 102 to low-order hydrocarbons 204. Like numbered items are as described with respect to FIG. 1. A hydrogen source 206 is also fed to the reactor 202 to provide the hydrogen needed for the conversion to hydrocarbons. The hydrogen source 206 may include hydrogen, water, or steam.

Any number of techniques known in the art may be used to convert the CO₂ 102 to low-order hydrocarbons 204, such as methane, ethane, ethanol, and the like. These techniques can include, for example, electrochemical, thermochemical, biocatalytic, or plasma-mediated processes for further use. For example, an electrochemical reduction of CO₂ 102 is used to generate CO, which is then used in a reverse water gas shift reaction with hydrogen to form methane and water. In another example, the Sabatier reaction is performed using the CO₂ 102 and a hydrogen feed to generate methane and water. In either the reverse water gas shift reaction or the Sabatier reaction, the catalyst can include nickel, ruthenium, or rhodium. In some embodiments, the catalyst is nickel.

In some embodiments, methane may be formed by electromethanogenesis, in which an electrochemical cell includes a cathode half-cell that uses a biological film, termed a biocathode, to reduce the CO₂ to methane with the input of protons and electrons to the biological film. A number of bacteria may be used to provide the biological film. In some examples, the biological film includes a colony of Methanobacterium palustre, an Archaeon. A proton conductive membrane, such as a sulfonated polytetrafluoroethylene membrane, separates the cathode half-cell from the paired anode half-cell. At the anode half-cell, water is oxidized, forming oxygen and the protons that are transferred to the cathode half-cell. The oxygen that is formed may be added to the oxidizer used for burning the fuel to power the process.

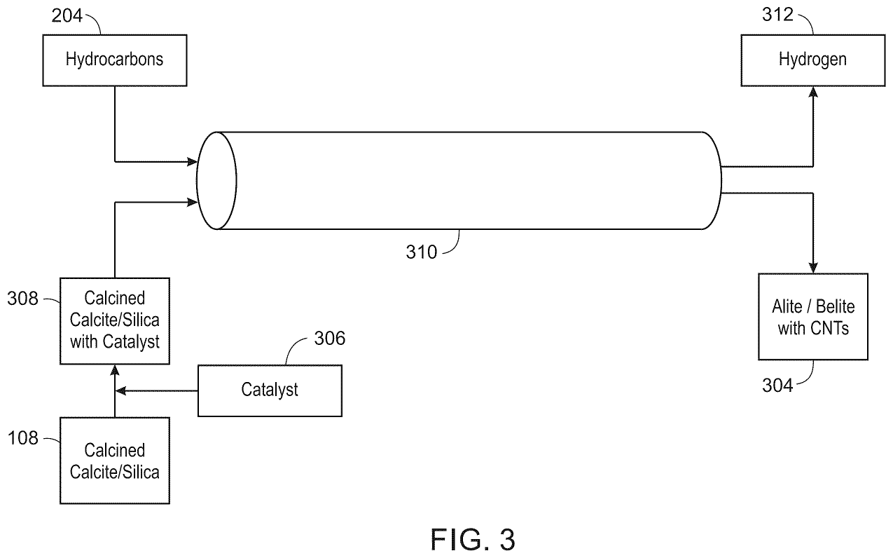

FIG. 3 is a schematic diagram of the production of CNTs on catalyst-impregnated, calcined cementitious precursors 302 to form CNT-reinforced cementitious materials 304. Like numbered items are as described with respect to FIGS. 1 and 2.

A catalyst 306 is loaded onto the calcined cementitious precursors 108. To load the catalyst 306, transition metal salts, such as halides, nitrates, acetates, or carbonates of Fe, Co, Ni, or Mo, are dissolved in a desired solvent, such as water, alcohols, or ethers. In some embodiments, the solution of the catalyst 306 is sprayed over the calcined cementitious precursors 108. In other embodiments, the calcined cementitious precursors 108 are fed through a bath of the catalyst 306, followed by drying the calcined cementitious precursors 108. Alternatively, the catalyst 306 can be deposited on the calcined cementitious precursors 108 using wet or dry chemistry methods such as sol-gel synthesis, hydrothermal/solvothermal synthesis, ball milling, spray coating, sputtering, or heat treatment.

The catalyst-treated, calcined cementitious precursors 308 are fed to a tube furnace 310 with the low-order hydrocarbons 204. In some embodiments, the heat of the tube furnace 310 combined with the low-order hydrocarbons 204 will reduce the metal of the catalyst 306, forming catalyst particles on the surface. In some embodiments, the tube furnace 310 is tilted from the material entry to the material exit, and material is moved along the tube furnace 310 by rotating the tube furnace 310.

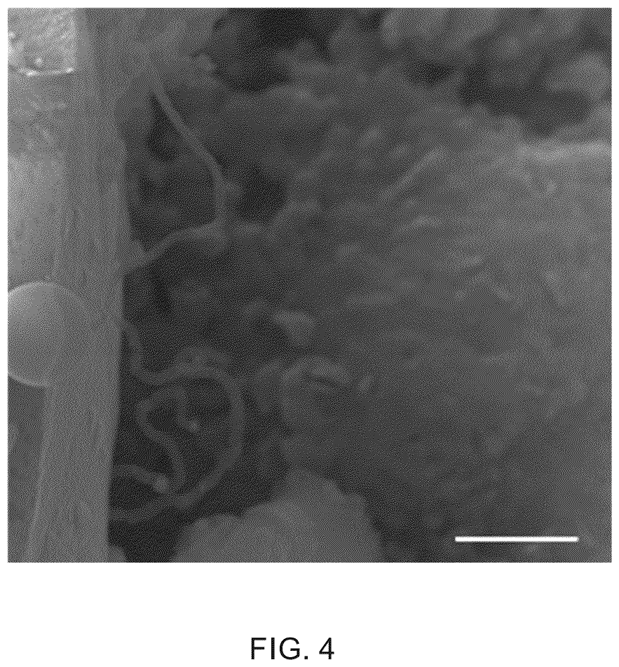

The low-order hydrocarbons 204 react on the surface of the catalyst particles to form the CNTs, releasing hydrogen 312. In addition to the hydrogen 312, the CNT-reinforced, cementitious materials 304, such as alite and belite, are formed, which may then be used to form CNT-reinforced cement as described herein. FIG. 4 is an electron micrograph of the CNTs formed on the cementitious material.

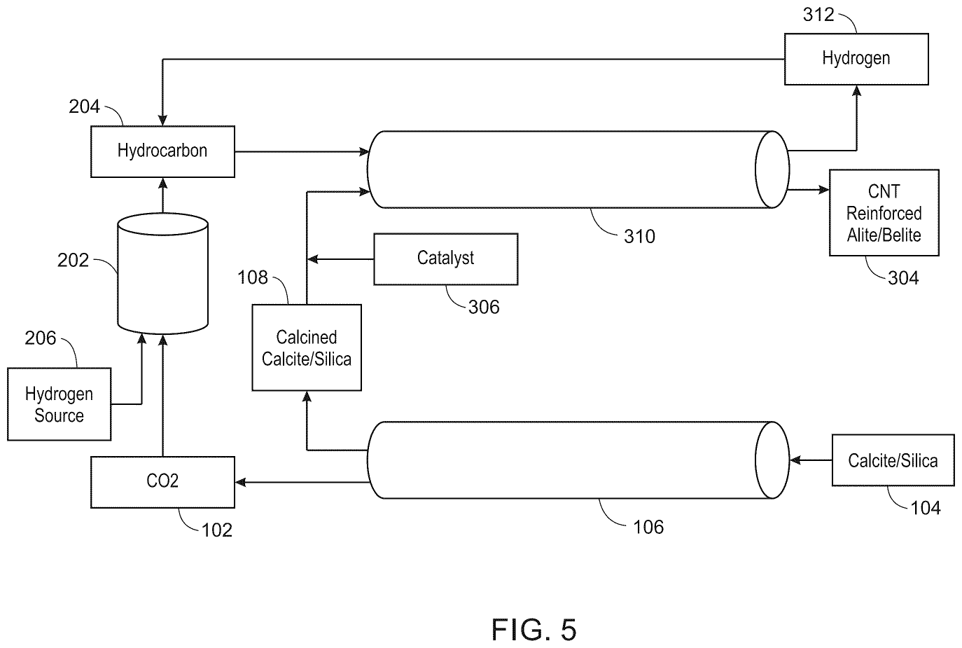

FIG. 5 is a schematic diagram of a combined system for making CNT reinforced CNT-reinforced, cementitious materials 304 by capturing carbon dioxide formed during the process. The system of FIG. 5 combines the systems discussed with respect to FIGS. 1, 2, and 3. Like numbered items are as described with respect to the previous figures.

In this embodiment, the cementitious precursors 104 are calcined in the tube reactor 106 as described with respect to FIG. 1. The CO₂ 102 formed during the calcination is captured, and used to form low-order hydrocarbons 204 in the reactor 202, as described with respect to FIG. 2. The emitted CO₂ 102 may be quantified to determine process efficiency, for example, using a GCMS. As described with respect to FIG. 2, electrochemical techniques, a Sabatier reaction, or other techniques are used to convert the CO₂ 102 into low order hydrocarbons or their derivatives, such as methane, ethane, acetylene, methanol, ethanol, and the like.

As described with respect to FIG. 3, the calcined cementitious precursors 108 are loaded with catalyst 306, and fed to the tube furnace 310. In the tube furnace 310, the CNTs are grown from the low-order hydrocarbons 204 at the catalyst particles on the surface of the calcined cementitious precursors by a catalytic chemical vapor deposition that occurs at a temperature of between about 600° C. to about 1100° C. The tube furnace 310 anneals the CNT-reinforced, cementitious precursors at a high temperature, e.g., about 1250° C. to about 1700° C., in the presence of an inert gas, such as nitrogen, helium, neon, argon, or krypton. The annealing produces the CNT-reinforced, cementitious materials 304, for example, including alite and belite. The CNT-reinforced, cementitious materials 304 can then be used to form CNT-reinforced cement.

During the growth of the CNTs, the hydrocarbon 204 is decomposed, resulting in the formation of hydrogen 312. The hydrogen 312 is separated out and may be used for various applications, such as a hydrogen fuel source or recycling back to the tube furnace 310 to assist in CNT growth. In some embodiments, the hydrogen 312 is recycled back to the reactor 202 as at least a portion of the hydrogen source 206 used to form methane and other low-order hydrocarbons from the carbon dioxide 102.

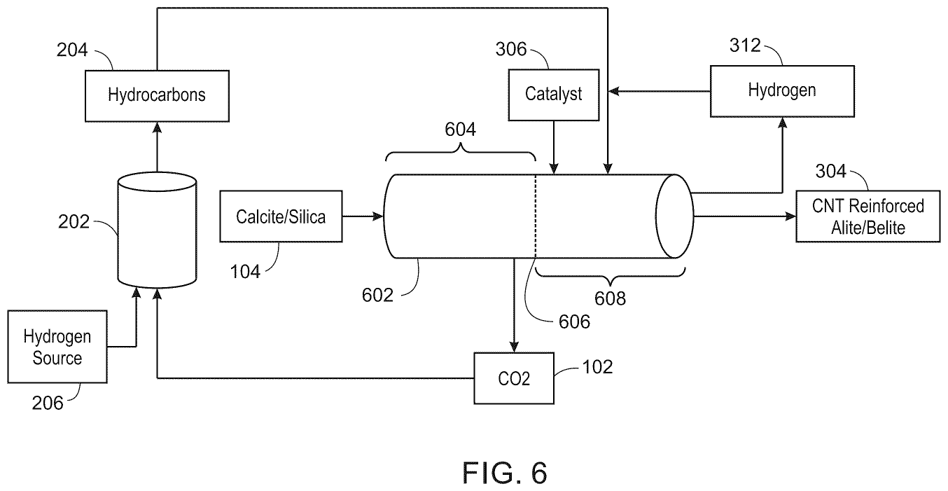

The reactor arrangement of FIG. 5 can be further optimized by combining the processes of both the tube reactor 106 and the tube furnace 310 into a single chamber. This is discussed with respect to FIG. 6.

FIG. 6 is a schematic diagram of a system for producing CNT-reinforced, cementitious materials 304. Like numbered items are as described with respect of the previous figures. In this configuration, the tube reactor 106 and tube furnace 310 are combined into a single tubular reactor 602. In some embodiments, the single tubular reactor 602 is tilted from the material entry to the material exit, and material is moved along the single tubular reactor 602 by rotating the single tubular reactor 602.

The material passes through a two-step heating process along the reactor. In a first portion 604 of the single tubular reactor 602, the temperature is controlled at about 600° C. to about 1100° C. to calcine the cementitious precursors, forming the calcined cementitious precursors 108 (calcined calcite/silica of FIG. 1). A screen 606, or other separating device, may slow the flow of solid from the first portion 604 to a second portion 608, for example, providing at least a partial vapor barrier. The CO₂ 102 formed in the calcination of the cementitious precursors can be substantially removed from the first portion 604, for example, and sent to the reactor 202 for the formation of the low-order hydrocarbons 204.

The solution of the catalyst 306 can be injected into the second portion 604, for example, downstream of the screen 602, along with the low-order hydrocarbons 204. The temperature and hydrocarbons will then reduce the metal salts, forming active catalyst particles on the calcined cementitious precursors. CNT will then form on the catalyst particles on the calcined cementitious precursors, as described with respect to FIG. 3. The calcined cementitious precursors with the CNTs are then heated to about 1250° C. to about 1700° C. to produce the CNT-reinforced, cementitious materials 304.

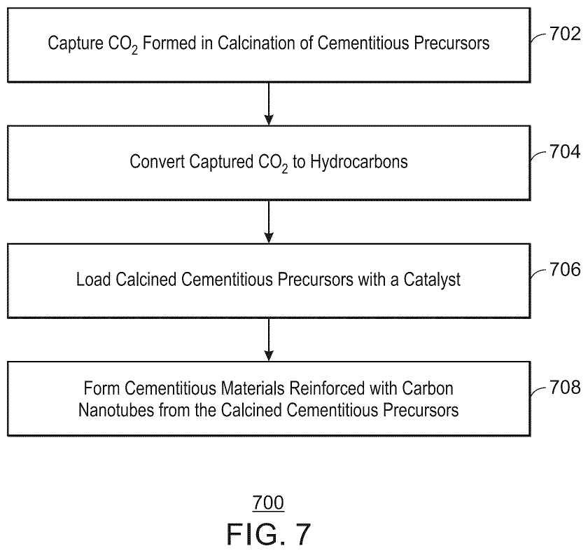

FIG. 7 is a process flow diagram of a method 700 for generating CNT-reinforced cementitious materials from carbon dioxide captured during the production of the cementitious materials. The method begins at block 702 with the capture of carbon dioxide formed in the calcining of cementitious precursors. At block 704, the captured CO₂ is converted to hydrocarbons, such as low order hydrocarbons that include methane, ethane, propane, and derivatives, such as methanol, ethanol, and the like. At block 706, a catalyst is loaded on the calcined cementitious precursors. Carbon nanotubes are produced on the calcined cementitious precursors, for example, by chemical vapor deposition of carbon at catalyst particles deposit on the cementitious precursors. At block 708, cementitious materials reinforced with the carbon nanotubes from the cementitious precursors are formed. The cementitious materials may be combined with aggregate and other materials, to form concrete.

### Embodiments

An embodiment described herein provides a method for generating carbon nanotube (CNT)-reinforced cementitious materials. The method includes capturing carbon dioxide formed in while calcining cementitious precursors, converting the carbon dioxide to hydrocarbons, producing CNTs on the calcined cementitious precursors from the hydrocarbons, and forming CNT-reinforced, cementitious materials from the calcined cementitious precursors comprising the CNTs.

In an aspect, the cementitious precursors include tricalcium silicate (C3S), dicalcium silicate (C2S), calcium aluminoferrite (C4AF), or calcium sulfate dihydrate (C1, gypsum), or any combinations thereof.

In an aspect, the method includes loading the calcined cementitious precursors with a catalyst.

In an aspect, the catalyst includes iron, cobalt, nickel, or molybdenum, or any combinations thereof. In an aspect, the method includes reducing the catalyst to form catalyst particles on the calcined cementitious precursors. In an aspect, the method includes forming the CNTs from the hydrocarbons by reaction of the hydrocarbons on a surface of the catalyst particles.

In an aspect, the method includes dissolving a transition metal salt in a solvent to form a catalyst solution, adding the catalyst solution to a cementitious precursor, and drying the catalyst solution to deposit the transition metal salt on the surface of the cementitious precursor.

In an aspect, the method includes capturing the carbon dioxide by absorbing the carbon dioxide in a trap. In an aspect, the trap includes carbonaceous materials, metal oxides, dichalcogenides, nitrides, or halides, or any combinations thereof.

In an aspect, the method includes converting the carbon dioxide to hydrocarbons by performing an electrochemical reaction on the carbon dioxide with water to produce carbon monoxide, and performing a reverse water gas shift reaction on the carbon monoxide with hydrogen to form methane. In an aspect, the method includes converting the carbon dioxide to hydrocarbons by performing a Sabatier reaction on the carbon dioxide to generate methane and water. In an aspect, the method includes converting the carbon dioxide to hydrocarbons by electromethanogenesis. In an aspect, the method includes electrochemically reducing the carbon dioxide to hydrocarbons.

In an aspect, the method includes forming hydrogen during formation of the carbon nanotubes, and recycling the hydrogen to form hydrocarbons from carbon dioxide. In an aspect, the method includes using a portion of the hydrogen to assist in forming the CNTs.

Another embodiment described herein provides a system for generating reinforced cementitious compositions. The system includes a tube reactor to calcine cementitious precursors, a tube furnace to form CNT-reinforced, cementitious materials from the calcined cementitious precursors and a hydrocarbon, a trap to capture carbon dioxide formed in the tube reactor, and a reactor to form the hydrocarbon from the carbon dioxide.

In an aspect, the tube reactor and the tube furnace are combined into a single tubular reactor.

In an aspect, the reactor includes a nickel catalyst to perform a Sabatier reaction to form the hydrocarbon from the carbon dioxide.

In an aspect, the reactor includes an electromethanogenesis reactor to produce the hydrocarbon from the carbon dioxide.

In an aspect, the hydrocarbon includes methane.

In an aspect, the trap includes carbonaceous materials, metal oxides, dichalcogenides, nitrides, or halides, or any combinations thereof.

Other implementations are also within the scope of the following claims.

What is claimed is:

1. A method for generating carbon nanotube (CNT)-reinforced cementitious materials, comprising:
capturing carbon dioxide formed in while calcining cementitious precursors;
converting the carbon dioxide to hydrocarbons;
producing CNTs on the calcined cementitious precursors from the hydrocarbons; and
forming CNT-reinforced, cementitious materials from the calcined cementitious precursors comprising the CNTs.

2. The method of claim 1, wherein the cementitious precursors comprise tricalcium silicate (C3S), dicalcium silicate (C2S), calcium aluminoferrite (C4AF), or calcium sulfate dihydrate (C1, gypsum), or any combinations thereof.

3. The method of claim 1, comprising loading the calcined cementitious precursors with a catalyst.

4. The method of claim 3, wherein the catalyst comprises iron, cobalt, nickel, or molybdenum, or any combinations thereof.

5. The method of claim 3, comprising reducing the catalyst to form catalyst particles on the calcined cementitious precursors.

6. The method of claim 3, comprising forming the CNTs from the hydrocarbons by reaction of the hydrocarbons on a surface of the catalyst particles.

7. The method of claim 3, comprising:
dissolving a transition metal salt in a solvent to form a catalyst solution;
adding the catalyst solution to a cementitious precursor; and
drying the catalyst solution to deposit the transition metal salt on the surface of the cementitious precursor.

8. The method of claim 1, comprising capturing the carbon dioxide by absorbing the carbon dioxide in a trap.

9. The method of claim 8, wherein the trap comprises carbonaceous materials, metal oxides, dichalcogenides, nitrides, or halides, or any combinations thereof.

10. The method of claim 1, comprising converting the carbon dioxide to hydrocarbons by:
performing an electrochemical reaction on the carbon dioxide with water to produce carbon monoxide; and
performing a reverse water gas shift reaction on the carbon monoxide with hydrogen to form methane.

11. The method of claim 1, comprising converting the carbon dioxide to hydrocarbons by performing a Sabatier reaction on the carbon dioxide to generate methane and water.

12. The method of claim 1, comprising converting the carbon dioxide to hydrocarbons by electromethanogenesis.

13. The method of claim 1, comprising electrochemically reducing the carbon dioxide to hydrocarbons.

14. The method of claim 1, comprising:
forming hydrogen during formation of the carbon nanotubes; and
recycling the hydrogen to form hydrocarbons from carbon dioxide.

15. The method of claim 14, comprising using a portion of the hydrogen to assist in forming the CNTs.

16. A system for generating reinforced cementitious compositions, comprising:
a tube reactor to calcine cementitious precursors;
a tube furnace to form CNT-reinforced, cementitious materials from the calcined cementitious precursors and a hydrocarbon;
a trap to capture carbon dioxide formed in the tube reactor; and
a reactor to form the hydrocarbon from the carbon dioxide.

17. The system of claim 16, wherein the tube reactor and the tube furnace are combined into a single tubular reactor.

18. The system of claim 16, wherein the reactor comprises a nickel catalyst to perform a Sabatier reaction to form the hydrocarbon from the carbon dioxide.

19. The system of claim 16, wherein the reactor comprises an electromethanogenesis reactor to produce the hydrocarbon from the carbon dioxide.

20. The system of claim 16, wherein the hydrocarbon comprises methane.

21. The system of claim 16, wherein the trap comprises carbonaceous materials, metal oxides, dichalcogenides, nitrides, or halides, or any combinations thereof.
